(19) United States
(12) Patent Application Publication
(10) Pub. No.: US 2011/0273668 A1
(43) Pub. Date: Nov. 10, 2011

(54) OPTICAL TOMOGRAPHIC IMAGING APPARATUS AND IMAGING METHOD FOR AN OPTICAL TOMOGRAPHIC IMAGE

(75) Inventor: Futoshi Hirose, Yokohama-shi (JP)

(73) Assignee: CANON KABUSHIKI KAISHA, Tokyo (JP)

(21) Appl. No.: 13/131,933

(22) PCT Filed: Dec. 18, 2009
(86) PCT No.: PCT/JP2009/071718

§ 371 Date: May 31, 2011

(30) Foreign Application Priority Data
Dec. 26, 2008 (JP) 2008-331925

(51) Int. Cl. A61B 3/14; A61B 3/00

(52) U.S. Cl. 351/206; 351/246

(57) ABSTRACT

Provided is an optical tomographic imaging apparatus capable of, when imaging a tomographic image of an object, monitoring an incident state represented by an incident position and an incident angle of a measuring beam group with respect to the object, causing the measuring beam group to form an image at a predetermined position of the object, and obtaining the tomographic image at high speed. The optical tomographic imaging apparatus is featured in that one of multiple beams emitted from a light source to be split and multiple beams emitted from multiple light sources are split into a measuring beam group and a reference beam group, and the optical tomographic imaging apparatus includes a monitoring device for obtaining a monitoring image of the object, thereby capable of monitoring an incident state represented by an incident position and an incident angle of the measuring beam group with respect to the object.

### TECHNICAL FIELD

The present invention relates to an optical tomographic imaging apparatus and an imaging method for an optical tomographic image, and more particularly, to an optical tomographic imaging apparatus and an imaging method for an optical tomographic image that are used for ophthalmological care or the like.

### BACKGROUND ART

Currently, there are various types of ophthalmological instruments using an optical instrument.

For instance, as an optical instrument for monitoring an eye, there are used various instruments such as an anterior eye part imaging instrument, a fundus camera, a confocal laser scanning ophthalmoscope (scanning laser ophthalmoscope: SLO), and the like.

In particular, an optical tomographic imaging apparatus that performs optical coherence tomography (OCT) utilizing an interference phenomenon of multi-wavelength light is an apparatus capable of obtaining a tomographic image of a sample with high resolution.

For this reason, the optical tomographic imaging apparatus is becoming an indispensable apparatus as an ophthalmological instrument for a specialist of retina in the outpatient field. Hereinbelow, the optical tomographic imaging apparatus is referred to as an OCT apparatus.

In the above-mentioned OCT apparatus, a measuring beam that is a low coherence beam is irradiated to a sample, and backscattered light from the sample can be measured with high sensitivity by using an interference system.

In addition, the OCT apparatus is capable of obtaining a tomographic image with high resolution by scanning the sample with the measuring beams.

This enables the OCT apparatus to image a tomographic image of a retina in the fundus of an object eye with high resolution, and hence the OCT apparatus is used widely for ophthalmological diagnosis of retina or the like.

In recent years, the OCT apparatus for ophthalmological use has been changing from a conventional time-domain method to a Fourier-domain method by which faster imaging is possible. High-speed imaging prevents blurring or missing of an image due to eye movement such as involuntary eye movement.

However, even with the Fourier-domain method capable of high-speed imaging, it is impossible to completely eliminate the blurring or the missing of an image due to the eye movement. Hence, further speed-up is desired.

In Japanese Patent Application Laid-Open No. 2006-195240, a microlens array and a Nipkow disk are used to realize a multi-beam OCT apparatus having multiple measuring beams. This OCT apparatus enables obtaining a tomographic image and a fluorescence tomographic image of a living body at high speed.

Japanese Patent No. 2,875,181 discloses an OCT apparatus including multiple light sources, an object beam image forming optical system provided in common for the multiple light sources, and multiple photosensors discretely disposed at positions corresponding to the positions of a reference beam image forming optical system provided in common and light sources.

In this OCT apparatus, data is obtained simultaneously at multiple points, and reference beams are shifted to obtain multi-point data, which enables data to be obtained at high speed.

Further, the OCT apparatus causes the measuring beams that are low coherence beams to form an image at a predetermined position of the retina, to thereby obtain a tomographic image.

However, there is a case where, due to a factor on the object eye side, such as difficulty in making the object eye remain at rest, it is difficult for the measuring beams to pass through the pupil and form an image at the predetermined position of the retina without being vignetted by the iris.

Specifically, in the OCT apparatus, if the measuring beams are vignetted by the iris, a ratio of the measuring beams reaching the predetermined position of the retina decreases, and accordingly, beams reflected from the retina may decrease. In this case, because there is an upper limit on power of the measuring beams for a safety reason, the contrast of the tomographic image to be obtained as a final result becomes low.

In particular, such a tendency becomes more conspicuous in a case where the beam diameter of the measuring beam is made larger so as to achieve an OCT apparatus having high resolution in a direction perpendicular to the optical axis, or in a case where a multi-beam OCT apparatus having multiple measuring beams is configured so as to achieve a high-speed OCT apparatus.

Japanese Patent Application Laid-Open No. 2002-174769 discloses an OCT apparatus for monitoring the inside of a biological sample, which is capable of high-resolution monitoring.

In this OCT apparatus, at the time of monitoring a sample, a beam diameter changing optical system is used to switch between a mode that enables high-resolution monitoring and a mode that enables wide-range monitoring, which therefore enables monitoring with a high S/N ratio.

As described above, when the OCT apparatus is used to monitor a fundus, there is a case where, due to a factor on the object eye side, such as difficulty in making the object eye remain at rest, it is difficult for the measuring beams to pass through the pupil and form an image at the predetermined position of the retina without irradiating the iris.

In particular, in a case where the multi-beam OCT apparatus having multiple measuring beams is configured so as to obtain a wide-range tomographic image at high speed, an influence thereof becomes more conspicuous.

In Japanese Patent Application Laid-Open No. 2006-195240 described above, the microlens array and the Nipkow disk are used to realize the multi-beam OCT apparatus, which is capable of high-speed imaging. However, Japanese Patent Application Laid-Open No. 2006-195240 gives no particular consideration to a measure against the above-mentioned factor on the object eye side, that is, the difficulty in making the object eye remain at rest, which is necessary at the time of monitoring a fundus. In Japanese Patent No. 2,875,181 described above, the OCT apparatus including the multiple light sources and the multiple photosensors is realized to enable high-speed imaging. However, Japanese Patent No. 2,875,181 also gives no particular consideration to a measure against the above-mentioned factor on the object eye side, that is, the difficulty in making the object eye remain at rest, which is necessary at the time of monitoring a fundus.

In Japanese Patent Application Laid-Open No. 2002-174769 described above, the beam diameter changing optical system is used to switch between the mode that enables high-resolution monitoring and the mode that enables wide-range monitoring, which therefore enables high-resolution monitoring.

However, Japanese Patent Application Laid-Open No. 2002-174769 also gives no particular consideration to a measure against the above-mentioned factor on the object eye side, that is, the difficulty in making the object eye remain at rest, which is necessary at the time of monitoring a fundus.

### DISCLOSURE OF THE INVENTION

In view of the above-mentioned problems, the present invention has an object to provide an optical tomographic imaging apparatus and an imaging method for an optical tomographic image, which are capable of, when imaging a tomographic image of an object, monitoring an incident state represented by an incident position and an incident angle of a measuring beam group with respect to the object, causing the measuring beam group to form an image at a predetermined position of the object, and obtaining the tomographic image at high speed.

The present invention provides an optical tomographic imaging apparatus configured as follows. The optical tomographic imaging apparatus is configured to:

split, multiple beams emitted from a light source or multiple beams emitted from multiple light sources, into a measuring beam group and a reference beam group, and guide the measuring beam group and the reference beam group to an object and a reference mirror, respectively; and

use a return beam group from the measuring beam group reflected or scattered by the object, and the reference beam group reflected by the reference mirror to image a tomographic image of the object,

the optical tomographic imaging apparatus comprising a monitoring device for obtaining a monitoring image of the object,

the monitoring device being capable of monitoring an incident state represented by an incident position and an incident angle of the measuring beam group with respect to the object.

According to the present invention, when the tomographic image of an object is imaged, the incident state represented by the incident position and the incident angle of the measuring beam group with respect to the object may be monitored, the measuring beam group may be caused to form an image at the predetermined position of the object, and the tomographic image may be obtained at high speed.

Further features of the present invention will become apparent from the following description of exemplary embodiments with reference to the attached drawings.

### BRIEF DESCRIPTION OF THE DRAWINGS

FIG. 1 is a diagram illustrating a configuration of an optical system of an OCT apparatus according to a first embodiment of the present invention.

FIGS. 2A, 2B, 2C, and 2D are diagrams for describing a method of obtaining a tomographic image by the OCT apparatus according to the first embodiment of the present invention.

FIGS. 3A and 3B are diagrams for describing a configuration of a measuring beam monitoring system of the OCT apparatus according to the first embodiment of the present invention.

FIGS. 4A, 4B, and 4C are diagrams for describing the configuration of the measuring beam monitoring system of the OCT apparatus according to the first embodiment of the present invention.

FIGS. 5A, 5B, and 5C are diagrams for describing the configuration of the measuring beam monitoring system of the OCT apparatus according to the first embodiment of the present invention.

FIGS. 6A and 6B are diagrams for describing the configuration of the measuring beam monitoring system of the OCT apparatus according to the first embodiment of the present invention.

FIGS. 7A, 7B, 7C, and 7D are diagrams for describing a method of adjusting a position of an object eye by the OCT apparatus according to the first embodiment of the present invention.

FIG. 8 is a flow chart of respective processes for describing a method of imaging an optical tomographic image according to the first embodiment of the present invention.

FIG. 9 is a diagram illustrating an overall configuration of an OCT apparatus according to a second embodiment of the present invention.

FIG. 10 is a diagram illustrating a configuration of an optical system of the OCT apparatus according to the second embodiment of the present invention.

FIG. 11 is a diagram illustrating a configuration of an OCT imaging portion of the OCT apparatus according to the second embodiment of the present invention.

### BEST MODE FOR CARRYING OUT THE INVENTION

Hereinbelow, an embodiment mode of the present invention is described.

In this embodiment mode, the above-mentioned configuration of the present invention may be applied to thereby configure, for example, the optical tomographic imaging apparatus (OCT apparatus) as described in the following (1) to (20).

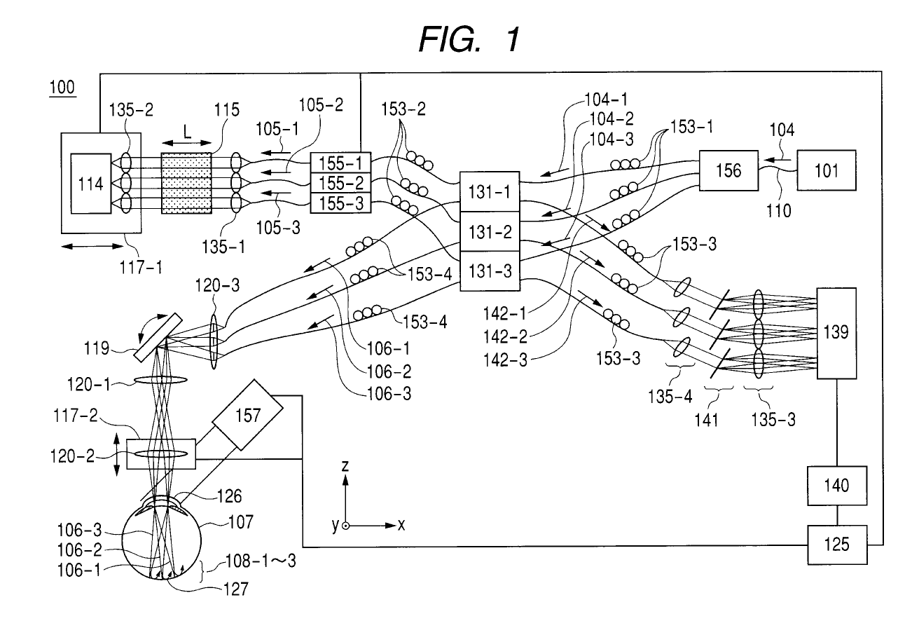

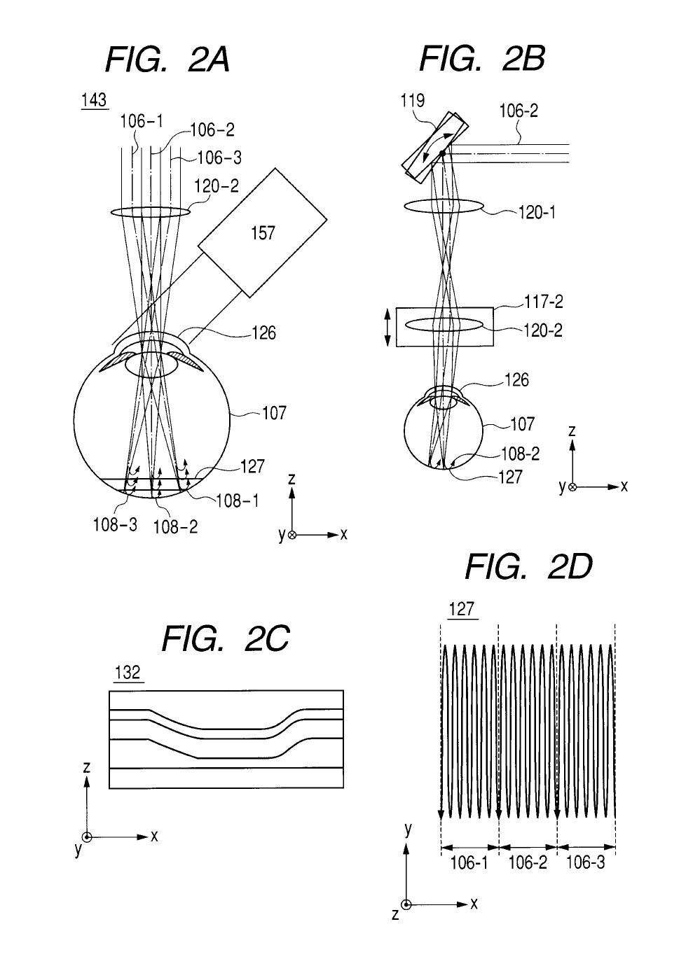

(1) As illustrated in FIG. 1, the optical tomographic imaging apparatus according to this embodiment mode is configured to: further split beams that have been emitted from a light source 101 and split into multiple beams, into a measuring beam group 106-1 to 106-3 and a reference beam group 105-1 to 105-3, the measuring beam group and the reference beam group including the split multiple beams; guide the measuring beam group and the reference beam group to an object 107 and a reference mirror 114, respectively; and use a return beam group 108-1 to 108-3 obtained from the measuring beam group reflected or scattered by the object, and the reference beam group reflected by the reference mirror to image a tomographic image (see FIG. 2C) of the object.

On this occasion, the optical tomographic imaging apparatus includes a monitoring device 157 for obtaining a monitoring image of the object, and the monitoring device is capable of monitoring an incident state represented by an incident position and an incident angle of the measuring beam group with respect to the object.

With this configuration, an optical tomographic imaging apparatus 100 including the measuring beam group constituted of multiple measuring beams monitors a state in which the object is irradiated with the measuring beam group.

As described above, with a configuration in which the optical tomographic imaging apparatus includes the monitoring device for obtaining a monitoring image, the state in which the object is irradiated with the measuring beam group may be recognized with ease.

As a result, positional relation between the measuring beam group and the object may be optimized with ease, enabling a wide-range tomographic image to be obtained at high speed.

(2) With a configuration in which the optical tomographic imaging apparatus includes a position recognizing device for recognizing the incident position of the measuring beam group based on the monitoring image obtained by the monitoring device, relative positions of the measuring beam group and the object may be recognized with ease, resulting in easier adjustment of the relative positions.

(3) With a configuration in which the monitoring device is disposed in proximity of an object eye that is the object, and is capable of monitoring a state in which an anterior eye part of the object eye is irradiated with the measuring beam group, the measuring beam group may enter the object eye in an optically optimal state.

(4) With a configuration in which the optical tomographic imaging apparatus includes an adjusting device (personal computer 125) capable of adjusting the relative positions of the measuring beam group and the object eye based on the monitoring image obtained by the monitoring device, the measuring beam group may enter the object eye optically appropriately.

(5) With a configuration in which the adjusting device is capable of adjusting an area of the anterior eye part, which is irradiated with the measuring beam group, to a minimum, the measuring beam group may enter the object eye optically appropriately.

(6) With a configuration in which the adjusting device is capable of increasing and decreasing a number of beams of the measuring beam group, an indicator for determining whether or not the relative positions of the measuring beam group and the object eye are closer to each other compared to optimal positions may be obtained.

(7) With a configuration in which a beam number increasing/decreasing device for the measuring beam group increases and decreases the number of beams of the measuring beam group to recognize the relative positions of the measuring beam group and the object eye, an indicator for determining how adjustment is made by using the adjustment device may be obtained.

(8) With a configuration in which the adjusting device is capable of increasing and decreasing a scanning range of the measuring beam group, the scanning range of the measuring beam group at the time of adjusting the relative positions of the measuring beam group and the object eye may be reduced, resulting in easier adjustment.

(9) With a configuration in which the adjusting device is capable of shifting a gaze with use of a fixation target (e.g. a fixation lamp) at which the object eye is to be guided, rotational movement of the object eye may be prompted, mainly. As a result, the measuring beam group may be caused to form an image at a predetermined position of a retina with ease.

(10) With a configuration in which the adjusting device is capable of shifting a face fixation unit for supporting a face of an examinee at a predetermined position, parallel shift of the object eye is enabled. As a result, the measuring beam group may be caused to form an image at the predetermined position of the retina with ease.

(11) With a configuration in which the adjusting device is capable of adjusting a measuring optical system for guiding the measuring beam group to the object, the measuring beam group may be adjusted so as to enter the object appropriately.

(12) With a configuration in which the optical tomographic imaging apparatus includes a recording device for recording the monitoring image and the tomographic image in association with each other, the state in which the measuring beam group enters the object may be recognized, which therefore allows discussion on the reliability of the obtained tomographic image.

(13) With a configuration in which the monitoring device includes a camera 157, the state in which the measuring beam group enters the anterior eye part may be monitored with ease.

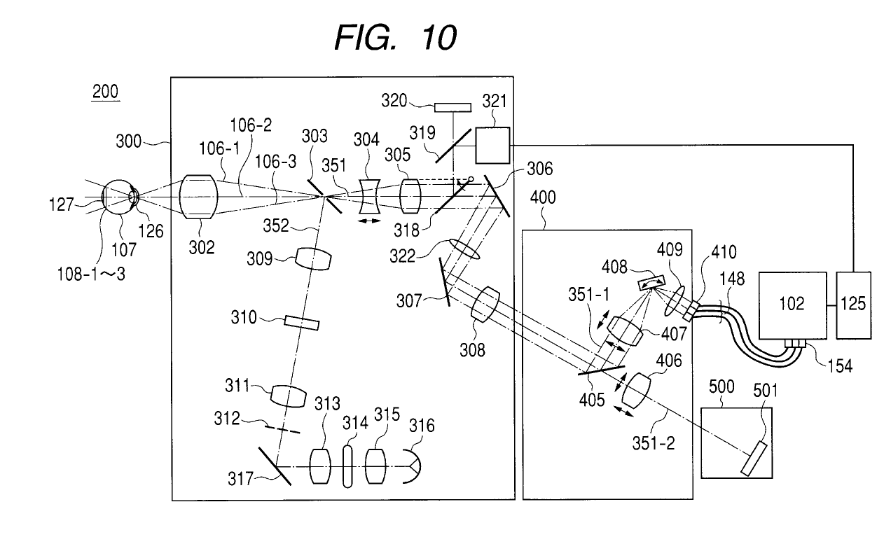

(14) With a configuration in which the monitoring device includes an area sensor (see 501 of FIG. 10), the state in which the measuring beam group enters the anterior eye part may be monitored with ease.

(15) With a configuration in which the monitoring device includes a confocal microscope, the state in which the measuring beams enter the anterior eye part may be monitored with ease.

(16) With a configuration in which the optical tomographic imaging apparatus includes an optical fiber forming at least one of the following optical paths, a compact optical tomographic imaging apparatus that is excellent in stability may be realized: an optical path for guiding the multiple beams obtained from the light source or the multiple beams emitted from the multiple light sources to a position at which the multiple beams are split into the measuring beam group and the reference beam group; an optical path for guiding the measuring beam group to the object; an optical path for guiding the return beam group to a photoelectric conversion circuit; and an optical path for guiding the reference beam group to the photoelectric conversion circuit.

(17) With a configuration in which the optical tomographic imaging apparatus for imaging a tomographic image of a fundus of the object eye includes a fundus camera main body portion 300 and a camera portion 500 for imaging a plane image of the fundus of the object eye, an apparatus having both functions of a fundus camera and an OCT apparatus may be realized.

Accordingly, an OCT apparatus with high space use efficiency and high profitability may be realized.

(18) With a configuration in which the fundus camera main body portion and the camera portion for imaging a plane image of the fundus are connectable to each other via an adapter 400, the function of the OCT apparatus may be realized by using an existing fundus camera.

(19) In the optical tomographic imaging apparatus described in any one of the above items (1) to (18), there is employed an imaging method for an optical tomographic image, in which a tomographic image of the object is imaged, including:

a first adjusting step of using a scanning range increasing/decreasing device to set the scanning range to be smaller than a desired imaging range;

a second adjusting step of using the monitoring device to monitor the state in which the anterior eye part is irradiated with the measuring beam group;

a third adjusting step of using the beam number increasing/decreasing device to recognize the relative positions of the measuring beam group and the object eye; and

a fourth adjusting step of using at least one of the face fixation unit, the fixation lamp, and the measuring optical system to adjust the relative positions of the measuring beam group and the object eye. As a result, the measuring beam group may be efficiently caused to form an image at the predetermined position of the retina of the object eye, resulting in efficient imaging.

(20) By automatically performing at least one of the first to fourth steps described above, the relative positions of the measuring beam group and the object eye may be efficiently adjusted.

### EMBODIMENTS

Next, embodiments of the present invention are described.

### First Embodiment

In a first embodiment, an OCT apparatus to which the present invention is applied is described. In this embodiment, in particular, an apparatus for imaging a tomographic image (OCT image) of an object eye is described.

The OCT apparatus described in this embodiment is a Fourier-domain OCT apparatus (Fourier Domain OCT), and is also a multi-beam OCT apparatus that has three measuring beams for fast imaging and is capable of obtaining three tomographic images simultaneously.

In this embodiment, the case where the OCT apparatus has three measuring beams is described, but the number of measuring beams may be increased depending on a predetermined imaging speed.

First, an overall schematic configuration of an optical system of the OCT apparatus according to this embodiment is described.

FIG. 1 is a diagram illustrating the overall schematic configuration of the optical system of the OCT apparatus according to this embodiment.

In FIG. 1, the OCT apparatus is represented by 100; a light source, 101; an emitted beam, 104; reference beams, 105-1, 105-2, and 105-3; measuring beams, 106-1, 106-2, and 106-3; multiplexed beams, 142-1, 142-2, and 142-3; the object eye, 107; return beams, 108-1, 108-2, and 108-3; a single mode fiber, 110; lenses, 120-1, 120-2, 120-3, 135-1, 135-2, 135-3, and 135-4; and a mirror, 114. Dispersion compensation glass is represented by 115; electrical stages, 117-1 and 117-2; an XY scanner, 119; and a personal computer, 125.

A cornea is represented by 126; a retina, 127; optical couplers, 131-1, 131-2, 131-3, and 156; a line camera, 139; a frame grabber, 140; a transmission grating, 141; polarization controllers, 153-1, 153-2, 153-3, and 153-4; fiber length adjusting devices, 155-1, 155-2, and 155-3; and a monitoring camera, 157.

As illustrated in FIG. 1, the OCT apparatus 100 of this embodiment forms a Michelson interference system as a whole.

In FIG. 1, the emitted beam 104 that is a beam emitted from the light source 101 is split by the optical coupler 156 into three emitted beams 104-1, 104-2, and 104-3. In this embodiment, a beam emitted from one light source is split into multiple beams to obtain multiple emitted beams. However, multiple light sources may be prepared to obtain multiple emitted beams.

Further, the emitted beams 104-1, 104-2, and 104-3 pass through the polarization controller 153-1, and are split, by the optical couplers 131-1, 131-2, and 131-3, into the reference beams 105-1, 105-2, and 105-3 and the measuring beams 106-1, 106-2, and 106-3, respectively, with an intensity ratio of 50:50.

The measuring beams 106-1, 106-2, and 106-3 are returned as the return beams 108-1, 108-2, and 108-3 that have been reflected or scattered by the retina 127 of the object eye 107 to be monitored. Then, the return beams 108-1, 108-2, and 108-3 are multiplexed with the reference beams 105-1, 105-2, and 105-3 by the optical couplers 131-1, 131-2, and 131-3.

After the reference beams 105-1, 105-2, and 105-3 and the return beams 108-1, 108-2, and 108-3 are multiplexed with each other, the resultant beams are dispersed according to the wavelengths by the transmission gratings 141, and input to the line camera 139. The line camera 139 converts a light intensity into a voltage for each position (wavelength), and the tomographic image of the object eye 107 is generated by using the voltage signals.

Next, the light source 101 and matters relevant thereto are described.

The light source 101 is a super luminescent diode (SLD), which is a typical low coherence light source.

The light source 101 has a wavelength of 830 nm and a bandwidth of 50 nm. Here, the bandwidth is an important parameter because the bandwidth affects the resolution of the obtained tomographic image in the optical axis direction.

In addition, the light source of an SLD type is used in this embodiment, but an amplified spontaneous emission (ASE) type or the like may also be used as long as the light source emits a low coherence beam. In addition, concerning the wavelength of light, near-infrared light is suitable because the light is used for measuring an eye.

Further, because the wavelength affects the resolution of the obtained tomographic image in the lateral direction, the wavelength is desirably as short as possible. Here, the wavelength is 830 nm. Depending on the measurement site to be monitored, another wavelength may be selected.

Next, optical paths of the reference beams 105-1, 105-2, and 105-3 are described.

The reference beams 105-1, 105-2, and 105-3 split by the optical couplers 131-1, 131-2, and 131-3 pass through the polarization controller 153-2, and the fiber length adjusting devices 155-1, 155-2, and 155-3. Then, the resultant beams are converted into parallel beams having a beam diameter of 1 mm by the lenses 135-1, and are then emitted.

Next, the reference beams 105-1, 105-2, and 105-3 pass through the dispersion compensation glass 115, and are condensed onto the mirror 114 by the lenses 135-2.

Next, the reference beams 105-1, 105-2, and 105-3 change the direction at the mirror 114, and are guided toward the optical couplers 131-1, 131-2, and 131-3 again.

Next, the reference beams 105-1, 105-2, and 105-3 pass through the optical couplers 131-1, 131-2, and 131-3, and are guided to the line camera 139.

Here, the dispersion compensation glass 115 compensates for dispersion that occurs when the measuring beams 106-1, 106-2, and 106-3 enter the object eye 107 and are reflected thereby, with respect to the reference beams 105-1, 105-2, and 105-3, respectively.

Here, assuming a value typical as an average diameter of the eyeball of Japanese people, L is set to 23 mm.

Further, the electrical stage 117-1 is capable of moving in directions indicated by arrows in the figure, which enables adjusting and controlling the optical path lengths of the reference beams 105-1, 105-2, and 105-3.

In addition, the electrical stage 117-1 may be controlled by the personal computer 125 at high speed.

Further, the fiber length adjusting devices 155-1, 155-2, and 155-3 are installed for the purpose of making fine adjustment on the respective fiber lengths, and are capable of adjusting the optical path lengths of the reference beams 105-1, 105-2, and 105-3 according to the respective measurement sites of the measuring beams 106-1, 106-2, and 106-3. The personal computer 125 may control the fiber length adjusting devices 155-1, 155-2, and 155-3.

Next, the optical paths of the measuring beams 106-1, 106-2, and 106-3 are described.

The measuring beams 106-1, 106-2, and 106-3 split by the optical couplers 131-1, 131-2, and 131-3 pass through the polarization controller 153-4, and are emitted as parallel beams having a beam diameter of 1 mm by the lens 120-3. The resultant beams are input to a mirror of the XY scanner 119.

Here, the XY scanner 119 is described as a single mirror for simple description, but actually, two mirrors of an X scan mirror and a Y scan mirror are disposed closely to each other so as to raster-scan the retina 127 in the direction perpendicular to the optical axis. Further, the lenses 120-1 and 120-3 are adjusted so that the center of each of the measuring beams 106-1, 106-2, and 106-3 is aligned with the rotation center of the mirror of the XY scanner 119.

The lenses 120-1 and 120-2, which constitute an optical system for scanning the retina 127 with the measuring beams 106-1, 106-2, and 106-3, have a role of scanning the retina 127 with the measuring beams 106-1, 106-2, and 106-3, with the vicinity of the cornea 126 set as a supporting point.

Here, focal lengths of the lenses 120-1 and 120-2 are 50 mm each.

Further, the electrical stage 117-2 is capable of moving in directions indicated by arrows, which enables adjusting and controlling the position of the lens 120-2 attached thereto. By adjusting the position of the lens 120-2, the measuring beams 106-1, 106-2, and 106-3 may be condensed at a specific layer of the retina 127 of the object eye 107 for monitoring.

Further, a case of the object eye 107 having a refractive error may also be handled. When the measuring beams 106-1, 106-2, and 106-3 enter the object eye 107, the measuring beams 106-1, 106-2, and 106-3 are reflected or scattered by the retina 127 to become the return beams 108-1, 108-2, and 108-3. Then, the return beams 108-1, 108-2, and 108-3 pass through the optical couplers 131-1, 131-2, and 131-3 to be guided to the line camera 139.

Here, the electrical stage 117-2 can be controlled by the personal computer 125 at high speed.

Next, a configuration of a measurement system of the OCT apparatus according to this embodiment is described.

The return beams 108-1, 108-2, and 108-3, which are beams reflected or scattered by the retina 127, and the reference beams 105-1, 105-2, and 105-3 are multiplexed with each other by the optical couplers 131-1, 131-2, and 131-3, respectively.

Then, the multiplexed beams 142-1, 142-2, and 142-3 are dispersed according to the wavelengths by the transmission gratings 141, and condensed by the lenses 135-3. Then, the intensity of light is converted into voltage for each position (wavelength) by the line camera 139.

Specifically, in association with the number of the measuring beams 106-1, 106-2, and 106-3, the line camera 139 monitors interference patterns of spectral regions along three wavelength axes.

A voltage signal group thus obtained is converted into digital values by the frame grabber 140. After that, the personal computer 125 performs data processing to form a tomographic image.

Here, the line camera 139 has 4,096 pixels, and uses 3,072 pixels thereof to obtain the intensity of the multiplexed beams 142-1, 142-2, and 142-3 for each of the wavelengths (divided into 1,024 positions).

Next, a method of obtaining a tomographic image by using the OCT apparatus is described.

Here, the method of obtaining a tomographic image (surface parallel to the optical axis) of the retina 127 is described with reference to FIGS. 2A, 2B, 2C, and 2D. Components identical to or corresponding to the components illustrated in FIG. 1 are denoted by the same reference numerals, and hence repetitive description thereof is omitted.

FIG. 2A illustrates a state in which the object eye 107 is monitored by the OCT apparatus 100.

As illustrated in FIG. 2A, the measuring beams 106-1, 106-2, and 106-3 pass through the cornea 126, enter the retina 127, and are reflected or scattered at various positions to become the return beams 108-1, 108-2, and 108-3. The return beams 108-1, 108-2, and 108-3 reach the line camera 139 with time delays corresponding to the respective positions.

Here, the bandwidth of the light source 101 is wide, and a spatial coherence length thereof is short. Therefore, if an optical path length for the reference beam path is substantially equal to an optical path length for the measuring beam path, the line camera 139 may detect the interference pattern. As described above, the line camera 139 obtains the interference pattern of the spectral region along the wavelength axis.

Next, considering characteristics of the line camera 139 and the transmission grating 141, the interference pattern, which is information along the wavelength axis, is converted into an interference pattern along an optical frequency axis for each of the multiplexed beams 142-1, 142-2, and 142-3.

Further, the converted interference pattern along the optical frequency axis is subjected to inverse Fourier transform so as to obtain information regarding a depth direction.

Further, for the sake of convenience, of the measuring beams, only the measuring beam 106-2 is illustrated in FIG. 2B. As illustrated in FIG. 2B, if the interference pattern is detected by driving the X-axis of the XY scanner 119, the interference pattern may be obtained for each position of the X-axis. In other words, information on the depth direction may be obtained for each position of the X-axis.

As a result, a two-dimensional distribution of intensities of the return beam 108-2 may be obtained with regard to an X-Z plane. FIG. 2C illustrates a tomographic image 132 thus obtained.

Inherently, the tomographic image 132 is constituted of intensities of the return beams 108 arranged in an array as described above, and is displayed as, for example, a gray scale image corresponding to the intensities. In FIG. 2C, only boundaries of the obtained tomographic image are emphasized and displayed.

Further, as illustrated in FIG. 2D, by controlling the XY scanner 119 to raster-scan the retina 127 with the measuring beams 106-1, 106-2, and 106-3, three tomographic images may be obtained simultaneously and successively. Here, the scanning is performed with a main scanning direction of the XY scanner 119 set as an X-axis direction and a sub-scanning direction thereof set as a Y-axis direction. As a result, multiple Y-Z plane tomographic images may be obtained. Note that, though a case where the measuring beams 106-1, 106-2, and 106-3 perform scanning without overlapping one another is described herein, overlapping scanning may also be performed for a purpose of registration of a tomographic image.

Next, with reference to FIG. 1, a configuration of a measuring beam monitoring system, which is a feature of the present invention, is described.

In the OCT apparatus 100, as described above, the measuring beams 106-1, 106-2, and 106-3 pass through the cornea 126, and then, the retina 127 is irradiated therewith. The monitoring camera 157 is installed for the purpose of monitoring a state in which the measuring beams 106-1, 106-2, and 106-3 pass through the cornea 126 and enter the retina 127.

Here, the monitoring camera 157 is installed on a right forward side of the object eye 107. However, as long as the monitoring camera 157 may monitor the vicinity of the cornea 126, the monitoring camera 157 may be located anywhere.

Further, by utilizing a monitoring image obtained by the monitoring device, an adjusting device configured so that relative positions of a measuring beam group and the object eye can be adjusted by the device may be configured as follows.

For example, with the monitoring camera 157 and the personal computer 125 electrically connected, the monitoring image obtained by the monitoring camera 157 is captured by the personal computer 125, is subjected to image processing or the like, and is used to adjust the relative positions of the OCT apparatus 100 and the object eye 107.

Further, the monitoring image may be displayed and stored in association with an OCT image. Here, in consideration of the wavelength of the measuring beams 106-1, 106-2, and 106-3, which is 830 nm, a near-infrared camera is used for the monitoring camera 157. Further, the near-infrared camera may be configured by combining a near-infrared area sensor and a lens.

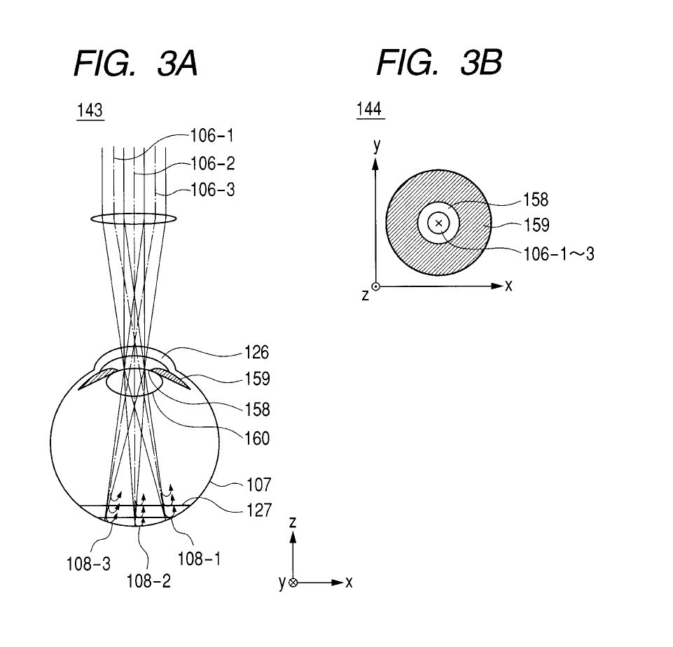

Next, with reference to FIGS. 3A and 3B, FIGS. 4A, 4B, and 4C, FIGS. 5A, 5B, and 5C, and FIGS. 6A and 6B, a monitoring image 144 obtained using the monitoring camera 157 is described.

Components identical or corresponding to the components illustrated in FIGS. 1, 2A, 2B, 2C, and 2D are denoted by the same reference numerals, and hence repetitive description thereof is omitted.

FIG. 3A is a schematic diagram 143 schematically illustrating a cross section of the object eye 107, which is a monitoring target.

Here, a pupil is represented by 158; an iris, 159; and a crystalline lens, 160. FIG. 3B illustrates the monitoring image 144.

Here, a state in which the object eye 107 is appropriately irradiated with the measuring beams 106-1, 106-2, and 106-3 is described.

Specifically, appropriate irradiation means a state in which the relative positions of the object eye 107 and the OCT apparatus 100 are adjusted so that the measuring beams 106-1, 106-2, and 106-3 pass through the pupil 158 without being vignetted by the iris 159, and that the measuring beams intersect in the vicinity of a surface of the crystalline lens 160.

Because the pupil 158 is the narrowest part in the optical paths of the measuring beams 106-1, 106-2, and 106-3, by adjusting an irradiation position according to the size of the pupil 158 as described above, wider measuring beams 106-1, 106-2, and 106-3 may be input onto the object eye 107, which is advantageous for achieving a higher resolution of the OCT apparatus 100.

FIG. 3B illustrates the monitoring image 144 for monitoring a state of the measuring beams 106-1, 106-2, and 106-3, with a focal point thereof adjusted to the vicinity of the surface of the crystalline lens 160.

Here, because the measuring beams 106-1, 106-2, and 106-3 pass through substantially the same position, the measuring beams 106-1, 106-2, and 106-3 are apparently recognized as one circle.

Here, a distance between the surface of the crystalline lens 160 and the lens 120-2 is 50 mm, which is equal to a focal length of the lens 120-2, hence aA mirror surface of the XY scanner 119 and the surface of the crystalline lens 160 have an optically conjugate relationship.

Next, a case where the relative positions of the object eye 107 and the OCT apparatus 100 are not appropriate is described.

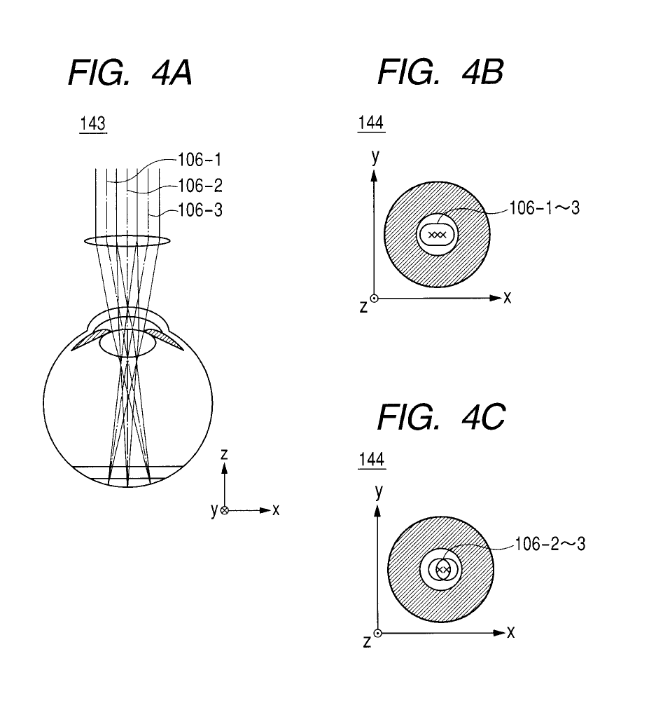

FIGS. 4A and 4B illustrate a case where the relative positions of the object eye 107 and the OCT apparatus 100 are closer to each other compared to optimal positions illustrated in FIG. 3A.

In this case, as can be seen in FIG. 4A, the measuring beams 106-1, 106-2, and 106-3 are monitored as being located in a wider area apparently as also illustrated in FIG. 4B.

Here, if the measuring beam 106-1 is shielded, a monitoring image 144 as illustrated in FIG. 4C is obtained, in which the measuring beams 106-2 and 106-3 are monitored in a +X direction compared to the case before shielding. Thus, it is understood that the relative positions of the object eye 107 and the OCT apparatus 100 are closer to each other compared to the optimal positions.

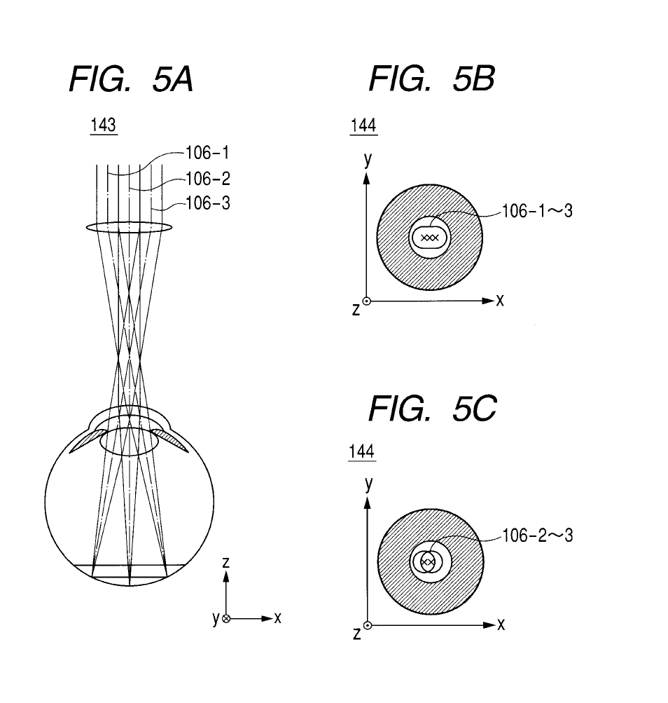

Further, as illustrated in FIG. 5A, in a case where the relative positions of the object eye 107 and the OCT apparatus 100 are distant from each other compared to the optimal positions illustrated in FIG. 3A, an monitoring image 144 as illustrated in FIG. 5B is obtained.

Similarly, if the measuring beam 106-1 is shielded, the monitoring image 144 as illustrated in FIG. 5C is obtained, in which the measuring beams 106-2 and 106-3 are monitored in a −X direction. Thus, it is understood that the relative positions of the object eye 107 and the OCT apparatus 100 are distant from each other compared to the optimal positions.

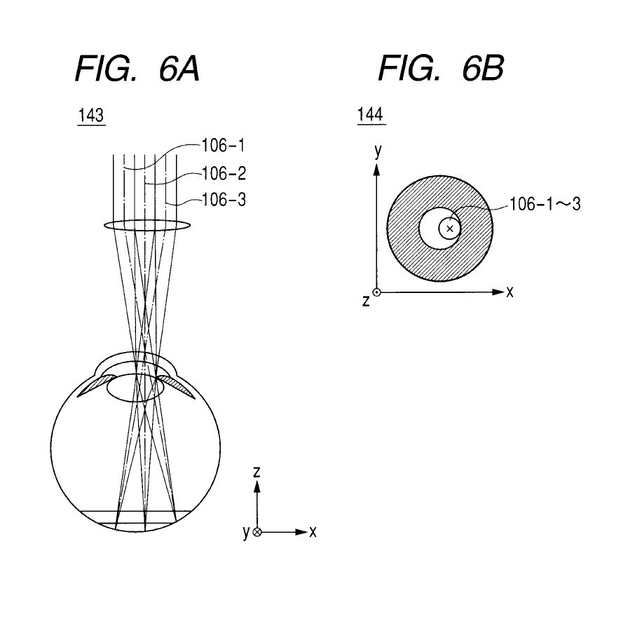

Further, as illustrated in FIG. 6A, in a case where the object eye 107 is displaced in the −X direction with respect to the OCT apparatus 100, a monitoring image 144 as illustrated in FIG. 6B is obtained, clearly indicating the above-mentioned situation.

As described above, in the cases where the relative positions of the object eye 107 and the OCT apparatus 100 are not appropriate, the above-mentioned optically conjugate relationship between the mirror surface of the XY scanner 119 and the surface of the crystalline lens 160 does not hold.

Accordingly, in states typified by FIGS. 4A, 5A, and 6A, the intensities of the return beams 108-1, 108-2, and 108-3 become smaller compared to the state of FIG. 3A. As a result, a S/N ratio of an interference signal for forming a tomographic image, which is described later, becomes lower.

In general, there is an upper limit for the energy of a measuring beam with which the retina is irradiated. Hence, in order to obtain a tomographic image suitable for diagnosis, it is important to input the measuring beams 106-1, 106-2, and 106-3 to the pupil 158 appropriately. Further, due to such a reason as being difficult to make an examinee remain at rest, even if the measuring beams 106-1, 106-2, and 106-3 unintentionally irradiate the iris 159, the monitoring image 144 may be used as means for assessing reliability of the obtained tomographic image.

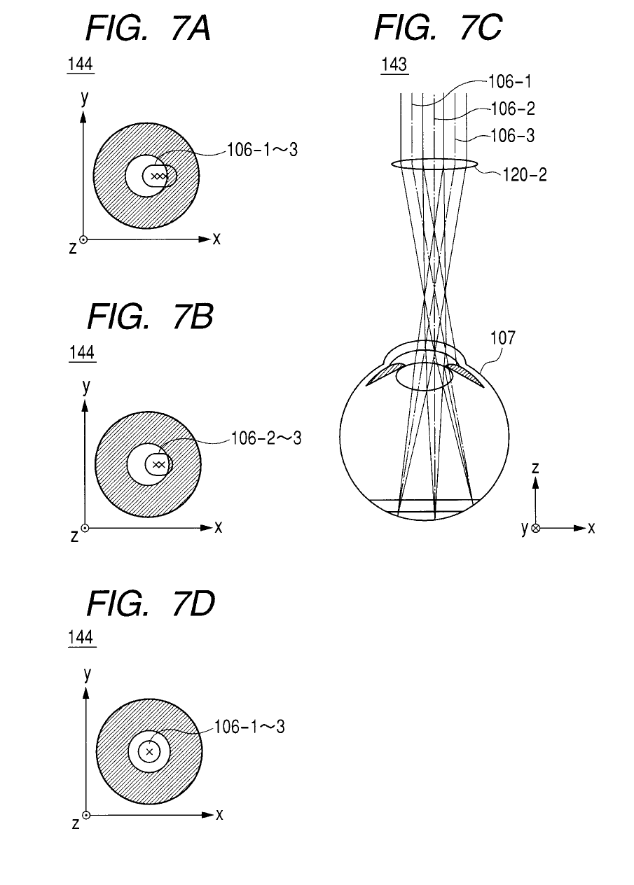

Next, referring mainly to FIGS. 7A, 7B, 7C, and 7D, an imaging method for an optical tomographic image, including adjusting the position of the object eye and imaging the optical tomographic image, which is a feature of the present invention, is described in detail.

Components identical to or corresponding to the components illustrated in FIG. 1, FIGS. 2A to 2D, FIGS. 3A and 3B, FIGS. 4A to 4C, FIGS. 5A to 5C, and FIGS. 6A and 6B are denoted by the same reference numerals, and hence repetitive description thereof is omitted.

In general, when the retina of the fundus is monitored, in consideration of safety, scanning is performed over the retina with measuring beams. The imaging method for an optical tomographic image according to this embodiment is performed by scanning the retina with the measuring beams, and a scanning range may be adjusted as necessary.

In the imaging method for an optical tomographic image, for example, the following processes of (1) to (4) are performed successively. Alternatively, the processes may be performed again after a while as necessary.

Further, by using a computer or the like, the following processes may be automatically performed.

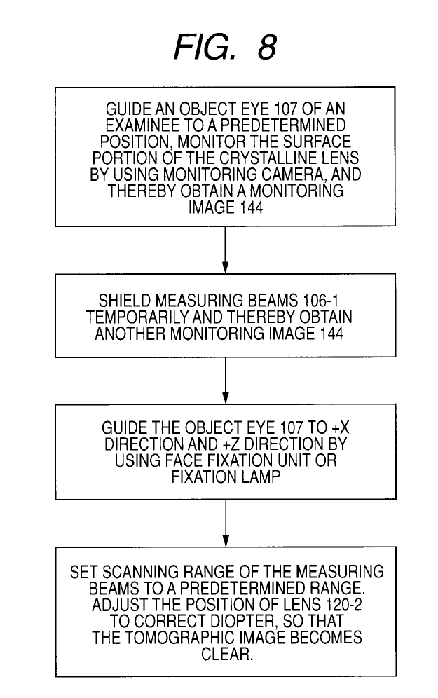

FIG. 8 is a flow chart of the respective processes for describing the method of imaging an optical tomographic image.

(1) The object eye 107 of the examinee is guided to a predetermined position, and then, the surface portion of the crystalline lens 160 is monitored by using the monitoring camera 157 (see FIG. 1) to obtain a monitoring image 144. Here, it is desired that the scanning range of the measuring beam be set to a relatively small range (FIG. 7A).

(2) The measuring beam 106-1 is temporarily shielded to obtain a monitoring image 144 (FIG. 7B). In the monitoring image 144, the measuring beams 106-2 and 106-3 are monitored on the side of the +X direction. Accordingly, the position of the object eye 107 is assumed to be as illustrated in FIG. 7C. Further, by using the personal computer 125, the monitoring image 144 may be subjected to image processing to quantify the intensities of the measuring beams 106-1, 106-2, and 106-3.

(3) By using a face fixation unit (not shown) or a fixation lamp (not shown), the object eye 107 is guided in the +X direction and in a +Z direction. By viewing the monitoring image 144, guidance and adjustment are appropriately made so that the measuring beams 106-1, 106-2, and 106-3 generate a minimum circle in appearance, and are located at the center of the pupil 158 (FIG. 7D).

(4) The scanning range of the measuring beams is set to a predetermined range. By adjusting the position of the lens 120-2, diopter correction is performed so that the tomographic image becomes clearer.

### Second Embodiment

In a second embodiment, an OCT apparatus to which the present invention applied is described.

In this embodiment, in particular, an apparatus for imaging a tomographic image (OCT image) and a fundus image (plane image) of an object eye is described.

In this embodiment, an OCT apparatus including an OCT imaging portion connected to a fundus camera via an adapter is described.

This embodiment describes an OCT apparatus with high space use efficiency and high profitability. Similarly to the first embodiment, the OCT apparatus described in this embodiment is an OCT apparatus of a Fourier-domain method, and is also a multi-beam OCT apparatus that has three measuring beams for fast imaging and is capable of obtaining three tomographic images simultaneously.

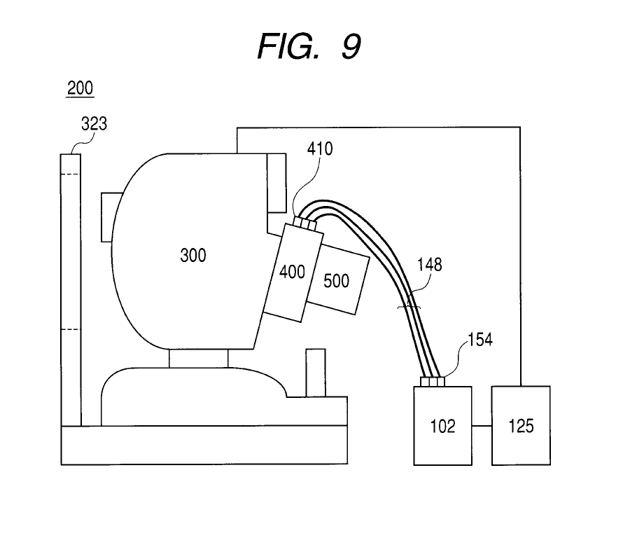

With reference to FIG. 9, an overall configuration of the OCT apparatus including the adapter of this embodiment is described. FIG. 9 is a side view of the OCT apparatus. An OCT apparatus 200 includes an OCT imaging portion 102, a fundus camera main body portion 300, an adapter 400, and a camera portion 500.

Here, the fundus camera main body portion 300, the adapter 400, and the camera portion 500 are optically connected to each other.

Here, the fundus camera main body portion 300 and the adapter 400 are supported so as to be relatively movable.

Therefore, optical adjustment may be performed roughly. In addition, the adapter 400 and the OCT imaging portion 102 are optically connected to each other via three single mode fibers 148. The adapter 400 and the OCT imaging portion 102 have three connectors 410 and three connectors 154, respectively. Therefore, the adapter 400 and the OCT imaging portion 102 are easily attachable to and detachable from each other. Further, a face fixation unit 323 fixes a chin and a forehead of an examinee so that the object eye is fixed for imaging.

Further, a personal computer 125 is used for creating and displaying the tomographic image.

Here, as the camera portion 500, a general-purpose digital single-lens reflex camera is used. The camera portion 500 is connected to the adapter 400 or the fundus camera main body portion 300 via a general-purpose camera mount.

Next, with reference to FIG. 10, a configuration of an optical system of the OCT apparatus including the adapter according to this embodiment is described.

In FIG. 10, the OCT apparatus 200 for measuring the object eye 107 includes the fundus camera main body portion 300, the adapter 400, the camera portion 500, and the OCT imaging portion 102. The OCT apparatus 200 is intended to obtain a tomographic image (OCT image) and a fundus image (plane image) of the retina 127 of the object eye 107 by using the OCT imaging portion 102 and the camera portion 500.

First, the fundus camera main body portion 300 is described.

An objective lens 302 is disposed so as to be opposed to the object eye 107, and, on the optical axis thereof, a perforated mirror 303 splits the optical path into an optical path 351 and an optical path 352.

The optical path 352 forms an illuminating optical system for illuminating the fundus of the object eye 107. In a lower portion of the fundus camera main body portion 300, there are disposed a halogen lamp 316 that is used for positioning the object eye 107, and a strobe tube 314 that is used for imaging the fundus of the object eye 107.

The fundus camera main body portion 300 further includes condenser lenses 313 and 315 and a mirror 317. Illuminating light emitted from the halogen lamp 316 and the strobe tube 314 is shaped into a ring-like light flux by a ring slit 312 and is reflected by the perforated mirror 303 so as to illuminate the fundus of the object eye 107.

The fundus camera main body portion 300 further includes lenses 309 and 311 and an optical filter 310.

The optical path 351 forms an imaging optical system for imaging the tomographic image and the fundus image of the fundus of the object eye 107. A focus lens 304 and an imaging lens 305 are disposed on the right side of the perforated mirror 303.

Here, the focus lens 304 is supported so as to be movable in the optical axis direction, and the personal computer 125 may control the position thereof. Next, the optical path 351 is guided to a fixation lamp 320 and a monitoring camera 321 via a quick return mirror 318.

Here, the quick return mirror 318 is designed to reflect and transmit a part of infrared light and to reflect visible light. Because the quick return mirror 318 is designed to reflect and transmit a part of infrared light, the fixation lamp 320, the monitoring camera 321, and the OCT imaging portion 102 may be used simultaneously.

In addition, a dichroic mirror 319 is designed to guide visible light in the direction to the fixation lamp 320 and guide infrared light in the direction to the monitoring camera 321.

Next, the optical path 351 is guided to the adapter 400 via a mirror 306, a field lens 322, a mirror 307, and a relay lens 308.

Here, the monitoring camera 321 monitors the vicinity of the cornea 126, which enables understanding a state in which the measuring beams 106-1, 106-2, and 106-3 enter the object eye 107, which is a characteristic of the present invention. In addition, with the use of the fixation lamp 320, the object eye 127 may be guided.

Next, a configuration of the optical system (adapter and camera portion) is described.

The largest function of the adapter 400 is to split the optical path 351 into an optical path 351-1 for imaging the tomographic image and an optical path 351-2 for imaging the fundus image via the dichroic mirror 405.

The adapter 400 further includes relay lenses 406 and 407, an XY scanner 408, and a collimator lens 409.

Further, here, the relay lenses 406 and 407 are supported in a movable manner so that the optical axis may be adjusted between the optical paths 351-1 and 351-2 by fine positional adjustment.

In addition, in FIG. 10, the XY scanner 408 is illustrated as a single mirror for simple description, but, actually, two mirrors of an X scan mirror and a Y scan mirror are disposed to be close to each other so as to raster-scan the retina 127 in the direction perpendicular to the optical axis.

In addition, the XY scanner 408 is controlled by the personal computer 125.

In addition, the optical axis of the optical path 351-1 is aligned with the rotation center of the two mirrors of the XY scanner 408.

In addition, with the use of the three connectors 410 for attaching three optical fibers, three measuring beams may be input from the OCT imaging portion 102 to the adapter 400, the fundus camera main body portion 300, and the object eye 107 in the stated order.

The camera portion 500 is a digital single-lens reflex camera for imaging the fundus image. The adapter 400 and the camera portion 500 are connected to each other via a general-purpose camera mount.

Hence, the adapter 400 and the camera portion 500 are easily attachable to and detachable from each other. The fundus image is generated on a surface of an area sensor 501.

Next, a configuration of the optical system (OCT portion) is described.

In this embodiment, the OCT imaging portion 102 has a configuration in which a part of the optical system is constituted of optical fibers for downsizing the apparatus.

The configuration of this embodiment is the same as the configuration of the first embodiment except for that the measuring optical system is constituted of the fundus camera main body portion 300.

Components identical to or corresponding to the components illustrated in FIG. 1 of the first embodiment are denoted by the same reference numerals, and hence repetitive description thereof is omitted.

First, an overall schematic configuration of an optical system of the OCT apparatus 102 according to this embodiment is described.

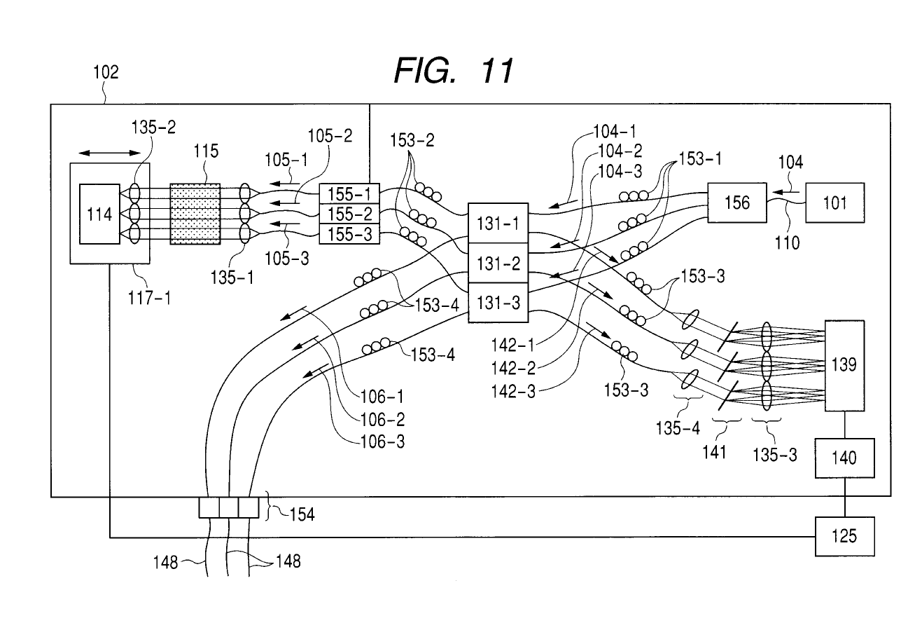

FIG. 11 is a diagram illustrating the overall schematic configuration of the optical system of the OCT apparatus 102 according to this embodiment.

In FIG. 11, the OCT imaging portion is represented by 102; a light source, 101; emitted beams, 104, 104-1, 104-2, and 104-3; reference beams, 105-1, 105-2, and 105-3; measuring beams, 106-1, 106-2, and 106-3; multiplexed beams, 142-1, 142-2, and 142-3; single mode fibers, 110 and 148; lenses, 135-1, 135-2, 135-3, and 135-4; and a mirror, 114.

Dispersion compensation glass is represented by 115; an electrical stage, 117-1; and a personal computer, 125. Optical couplers are represented by 131-1, 131-2, 131-3, and 156; a line camera, 139; a frame grabber, 140; transmission gratings, 141; polarization controllers, 153-1, 153-2, 153-3, and 153-4; and fiber length adjusting devices, 155-1, 155-2, and 155-3.

As illustrated in FIG. 11, the OCT apparatus 100 of this embodiment constitutes a Michelson interference system as a whole.

In FIG. 11, the emitted beam 104 that is a beam emitted from the light source 101 is split by the optical coupler 156 into the three emitted beams 104-1, 104-2, and 104-3.

Further, the emitted beams 104-1, 104-2, and 104-3 pass through the polarization controller 153-1, and are split, by the optical couplers 131-1, 131-2, and 131-3, into the reference beams 105-1, 105-2, and 105-3 and the measuring beams 106-1, 106-2, and 106-3, respectively, with an intensity ratio of 50:50.

The measuring beams 106-1, 106-2, and 106-3 are returned as return beams 108-1, 108-2, and 108-3 that have been, via the connector 154, the adapter 400, and the fundus camera main body portion 300, reflected or scattered by the retina 127 of the object eye 107 to be monitored (FIG. 10). Then, the return beams 108-1, 108-2, and 108-3 are multiplexed with the reference beams 105-1, 105-2, and 105-3 by the optical couplers 131-1, 131-2, and 131-3.

After the reference beams 105-1, 105-2, and 105-3 and the return beams 108-1, 108-2, and 108-3 are multiplexed with each other, the resultant beams are dispersed according to the wavelengths by the transmission gratings 141, and input to the line camera 139. The line camera 139 converts a light intensity into a voltage for each position (wavelength), and the tomographic image of the object eye 107 is generated by using the voltage signals.

Next, the light source 101 and matters relevant thereto are described.

The light source 101 is a super luminescent diode (SLD), which is a typical low coherence light source. The light source 101 has a wavelength of 830 nm and a bandwidth of 50 nm.

Here, the bandwidth is an important parameter because the bandwidth affects the resolution of the obtained tomographic image in the optical axis direction.

In addition, the light source of an SLD type is used in this embodiment, but an amplified spontaneous emission (ASE) type or the like may also be used as long as the light source emits a low coherence beam.

In addition, concerning the wavelength of light, near-infrared light is suitable because the light is used for measuring an eye. Further, because the wavelength affects the resolution of the obtained tomographic image in the lateral direction, the wavelength is desirably as short as possible. Here, the wavelength is 830 nm. Depending on the measurement site to be monitored, another wavelength may be selected.

Next, optical paths of the reference beams 105-1, 105-2, and 105-3 are described.

The reference beams 105-1, 105-2, and 105-3 split by the optical couplers 131-1, 131-2, and 131-3 pass through the polarization controller 153-2 and the fiber length adjusting devices 155-1, 155-2, and 155-3. Then, the resultant beams are converted into parallel beams having a beam diameter of 1 mm by the lenses 135-1, and are then emitted.

Next, the reference beams 105-1, 105-2, and 105-3 pass through the dispersion compensation glass 115, and are condensed onto the mirror 114 by the lenses 135-2.

Next, the reference beams 105-1, 105-2, and 105-3 change the direction at the mirror 114, and are guided toward the optical couplers 131-1, 131-2, and 131-3 again.

Next, the reference beams 105-1, 105-2, and 105-3 pass through the optical couplers 131-1, 131-2, and 131-3, and are guided to the line camera 139.

The dispersion compensation glass 115 compensates for dispersion that occurs when the measuring beams 106-1, 106-2, and 106-3 travel to and from the object eye 107, with respect to the reference beams 105-1, 105-2, and 105-3, respectively.

Here, assuming a value typical as an average diameter of the eyeball of Japanese people, L is set to 23 mm.

Further, the electrical stage 117-1 is capable of moving in directions indicated by arrows, which enables adjusting and controlling the optical path lengths of the reference beams 105-1, 105-2, and 105-3.

In addition, the electrical stage 117-1 can be controlled by the personal computer 125 at high speed.

Further, the fiber length adjusting devices 155-1, 155-2, and 155-3 are installed for the purpose of making fine adjustment on the respective fiber lengths, and are capable of adjusting the optical path lengths of the reference beams 105-1, 105-2, and 105-3 according to the respective measurement sites of the measuring beams 106-1, 106-2, and 106-3. The personal computer 125 can control the fiber length adjusting devices 155-1, 155-2, and 155-3.

Next, the optical paths of the measuring beams 106-1, 106-2, and 106-3 are described.

The measuring beams 106-1, 106-2, and 106-3 split by the optical couplers 131-1, 131-2, and 131-3 pass through the polarization controller 153-4. Then, via the connectors 154, the single mode fibers 148, the adapter 400, and the fundus camera main body portion 300, the measuring beams 106-1, 106-2, and 106-3 are guided to the retina 127 of the object eye 107 (see FIG. 10).

After entering the object eye 107, the measuring beams 106-1, 106-2, and 106-3 are reflected or scattered by the retina 127 to become the return beams 108-1, 108-2, and 108-3.

The return beams 108-1, 108-2, and 108-3 are guided to the optical couplers 131-1, 131-2, and 131-3 again via the fundus camera main body portion 300, the adapter 400, the connectors 410, the single mode fibers 148, and the connectors 154 in the stated order.

The above-mentioned reference beams 105-1, 105-2, and 105-3 and the above-mentioned return beams 108-1, 108-2, and 108-3 are multiplexed with each other by the optical couplers 131-1, 131-2, and 131-3, respectively, and then split in half.

Then, the multiplexed beams 142-1, 142-2, and 142-3 are dispersed according to the wavelengths by the transmission gratings 141, and condensed by the lenses 135-3. Then, the intensity of light is converted into voltage for each position (wavelength) by the line camera 139.

Specifically, an interference pattern of the spectral region along the wavelength axis is monitored on the line camera 139.

Next, a configuration of a measurement system of the OCT apparatus according to this embodiment is described.

The OCT imaging portion 102 is capable of obtaining a tomographic image (OCT image) generated based on the intensities of interference signals from a Michelson interference system.

To give further description of the measurement system, the return beams 108-1, 108-2, and 108-3 reflected or scattered by the retina 127 are multiplexed with the reference beams 105-1, 105-2, and 105-3 by the optical couplers 131-1, 131-2, and 131-3, respectively. Then, the multiplexed beams 142-1, 142-2, and 142-3 are dispersed according to the wavelengths by the transmission gratings 141, and condensed by the lenses 135-3. The intensity of light is converted into voltage for each position (wavelength) by the line camera 139.

Specifically, in association with the number of the measuring beams 106-1, 106-2, and 106-3, the line camera 139 monitors interference patterns of spectral regions along three wavelength axes.

A voltage signal group thus obtained is converted into digital values by the frame grabber 140. After that, the personal computer 125 performs data processing to form a tomographic image.

Here, the line camera 139 has 4,096 pixels, and uses 3,072 pixels thereof to obtain the intensity of each of the respective wavelengths (divided into 1,024 positions) of the multiplexed beams 142-1, 142-2, and 142-3.

Next, a method of obtaining a tomographic image is described.

The method of obtaining a tomographic image by using the OCT apparatus is substantially the same as in the first embodiment, and hence description thereof is omitted.

The OCT apparatus 200 controls the XY scanner 408, and, by obtaining the interference pattern by the line camera 139, the tomographic image of the retina 127 may be obtained (FIG. 10).

Next, a configuration of a measuring beam monitoring system is described.

The configuration of the measuring beam monitoring system, which is a characteristic of the present invention, is substantially the same as in the first embodiment except for that the monitoring camera 321 is installed inside the fundus camera main body portion 300, and hence repetitive description thereof is omitted.

The OCT apparatus 200 uses the monitoring camera 321 installed inside the fundus camera main body portion 300 to monitor the measuring beams 106-1, 106-2, and 106-3 in the vicinity of the cornea 126, which enables adjusting the relative positions of the OCT apparatus 200 and the object eye 107.

Further, the adjustment may be performed by using the fixation lamp 320, the face fixation unit 323, the personal computer 125, and the like.

### Other Embodiments

Aspects of the present invention may also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (e.g., computer-readable medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-331925, filed Dec. 26, 2008, which is hereby incorporated by reference herein in its entirety.

1. An optical tomographic imaging apparatus that obtains at least a tomographic image of an subject's eye based on a plurality of combined beams obtained by combining a plurality of return beams from the subject's eye that is irradiated with a plurality of measuring beams and a plurality of reference beams each of which respectively correspond to each of the plurality of measuring beams, the optical tomographic imaging apparatus comprising:
an irradiating device that irradiates an anterior eye part of the subject's eye with the plurality of measuring beams;
a monitoring device that obtains a monitoring image of the subject's eye; and
an adjusting device that obtains information of irradiated ranges where the anterior eye part is irradiated with the plurality of measuring beams by said irradiating device from the monitoring image, and that adjusts each of the irradiated ranges to be a predetermined overlapped state based on the information of the irradiated ranges.

2-4. (canceled)

5. The optical tomographic imaging apparatus according to claim 1, wherein said adjusting device is configured in at least one of the following manners that:
an area of the anterior eye part, which is irradiated with the plurality of measuring beams, is adjusted to a minimum;
the relative positions of the plurality of measuring beams and the subject's eye are recognized by increasing and decreasing a number of beams of the plurality of measuring beams;
a scanning range of the plurality of measuring beams is increased and decreased;
a gaze is shifted by using a fixation target at which the subject's eye is to be guided; and
a face fixation unit that supports a face of an examinee at a predetermined position is shifted.

6. The optical tomographic imaging apparatus according to claim 1, wherein said adjusting device adjusts relative positions of the plurality of measuring beams and the subject's eye.

7. The optical tomographic imaging apparatus according to claim 1, further comprising a recording device that records the monitoring image and the tomographic image in association with each other.

8. The optical tomographic imaging apparatus according to claim 1, wherein said monitoring device comprises at least one of a camera, an area sensor, and a confocal microscope.

9. The optical tomographic imaging apparatus according to claim 1, further comprising an optical fiber forming at least one of the following optical paths:
an optical path that guides multiple beams obtained from a light source or multiple beams emitted from multiple light sources to a position at which the multiple beams are split into the plurality of measuring beams and the plurality of reference beams;
an optical path that guides the plurality of measuring beams to the subject's eye;
an optical path that guides the plurality of return beams to a photoelectric conversion circuit; and
an optical path that guides the plurality of reference beams to the photoelectric conversion circuit.

10. (canceled)

11. A non-transitory computer-readable recording medium that has the program that executes the method according to claim 12 recorded thereon.

12. A method for taking an optical tomographic image by using the optical tomographic imaging apparatus according to claim 5 to thereby take a tomographic image of the subject's eye, the method comprising:
a first adjusting step of setting the scanning range to be smaller than a predetermined imaging range;
a second adjusting step of using the monitoring device to monitor a state in which the anterior eye part is irradiated with the plurality of measuring beams;
a third adjusting step of increasing/decreasing the number of the plurality of measuring beams to recognize the relative positions of the plurality of measuring beams and the subject's eye; and
a fourth adjusting step of using at least one of the face fixation unit, a fixation lamp, and a measuring optical system to adjust the relative positions of the plurality of measuring beams and the subject's eye.

13. An optical tomographic imaging apparatus that obtains at least a tomographic image of an subject's eye based on a plurality of combined beams obtained by combining a plurality of return beams from the subject's eye that is irradiated with a plurality of measuring beams and a plurality of reference beams each of which respectively correspond to each of the plurality of measuring beams, the optical tomographic imaging apparatus comprising:
an irradiating device that irradiates an anterior eye part of the subject's eye with the plurality of measuring beams;
an obtaining device that obtains information of the irradiated ranges where the anterior eye part is irradiated with the plurality of measuring beams by said irradiating device; and
an adjusting device that adjusts each of the irradiated ranges to be a predetermined overlapped state based on the information of the irradiated ranges.

14. The optical tomographic imaging apparatus according to claim 13, wherein the information of the irradiated ranges is information of an overlapped area of the irradiated ranges, and said adjusting device increases the overlapped area in order to adjust to the predetermined overlapped state.

15. The optical tomographic imaging apparatus according to claim 13, wherein the information of the irradiated ranges is information of a distance between an approximate center of the irradiated ranges, and said adjusting device decreases the distance in order to adjust to the predetermined overlapped state.

16. The optical tomographic imaging apparatus according to claim 13, wherein the predetermined overlapped state is a state that an optical axis of the plurality of measuring beams intersect at an approximate center of the anterior eye part of the subject's eye.

17. The optical tomographic imaging apparatus according to claim 13, wherein said adjusting device comprises a distance shifting device that shifts the distance between the irradiating device and the anterior eye part of the subject's eye.

18. The optical tomographic imaging apparatus according to claim 13, wherein said obtaining device comprises a monitoring image obtaining device that obtains a monitoring image of the anterior eye part of the subject's eye, and said obtaining device obtains information of the irradiated ranges by analyzing the monitoring image.
